(19) United States
(12) Patent Application Publication
(10) Pub. No.: US 2023/0321161 A1
(43) Pub. Date: Oct. 12, 2023

(54) A COMPOSITON COMPRISING MICROCAPSULES

(71) Applicant: NUABIOME LIMITED, DUBLIN (IE)

(72) Inventors: Sinead Bleiel, Dublin (IE); Maja Severic, Cork (IE); Mark Lee, Cork (IE)

(21) Appl. No.: 18/020,234

(22) PCT Filed: Jul. 27, 2021
(86) PCT No.: PCT/EP2021/071086

§ 371 Date: Feb. 7, 2023

(30) Foreign Application Priority Data
Jul. 27, 2020 (GB) 2011598.6

(51) Int. Cl. A61K 35/741; A61K 9/50; A61K 9/48

(52) U.S. Cl. CPC A61K 35/741; A61K 9/4866; A61K 9/50; A61K 9/5089

(57) ABSTRACT

A composition comprising microcapsules in an oral dosage form is described. The oral dosage form comprises gastric-resistant ileal-sensitive microcapsules comprising a matrix and active agent such as probiotic bacterium, contained within the matrix, in which the matrix comprises denatured whey protein, and in which the microcapsules are be coldgelated and vacuum dried microcapsules, and thus are subject to less heat treatment than conventional probiotic-containing microparticles. The microcapsules may be formed by extrusion through a single or double nozzle and are vacuum dried to a water activity (Aw) of 0.30 or less. The microcapsules may be subjected to two separate vacuum drying steps to further reduce the water activity and provide microcapsules with greater stability against moisture, humidity and thermal processes such as pasteurisation and Ultra High Temperatures (i.e. UHT)

### FIELD OF THE INVENTION

The present invention relates to a composition comprising microcapsules suitable for delivery of active agents such a probiotic bacteria to the gut of a subject via an oral route.

### SUMMARY OF THE INVENTION

The Applicant provides an oral dosage form that can be used to deliver active agents such as probiotics via the oral route. The oral dosage form comprises microcapsules having a protein matrix and active agent contained and protected within the matrix. The protein is generally whey protein, or another protein containing β-lactoglobulin. The matrix is resistant to gastric conditions and configured to break up in the ileum, releasing the contents gradually over a period of time.

The Applicant has also discovered that microcapsules produced by cold gelation (e.g. extrusion into a curing/gelation) bath), and drying by vacuum drying, are ideal for delivering heat-sensitive active agent such as probiotics via the oral route, as the cold gelation and vacuum drying processes avoid the high heat processes conventionally used for producing microparticles, resulting in less thermal damage to the probiotics and greater payload of active probiotic. Two vacuum drying steps are also described which allows microcapsules having a low water activity (e.g. 0.25 or less) to be produced, with greater stability against moisture, humidity and thermal processes such as pasteurisation and Ultra High Temperatures (i.e. UHT)

In a first aspect, the invention provides a composition such as an oral dosage form comprising microcapsules comprising a matrix and an active agent contained within the matrix, in which the matrix comprises or consists essentially of denatured protein.

In one embodiment, the microcapsules are cold-gelated and vacuum dried.

In one embodiment, the microcapsules are vacuum dried in two separate vacuum drying steps.

In any embodiment, the matrix consists essentially of denatured whey protein.

In any embodiment, the microcapsules have a water activity (Aw) of less than 0.25.

In any embodiment, the microcapsules have a water activity (Aw) of 0.20 or less.

In any embodiment, the microcapsules have a water activity (Aw) of 0.15 or less.

In any embodiment, the microcapsules have a water activity (Aw) of at least 0.05 or 0.10.

In any embodiment, at least 50%, 60%, 70%, 80%, 90%, 95% or 99% by weight of the matrix is protein.

In any embodiment, the microcapsules are substantially free of a polysaccharide based gelling agent such as alginate.

The active agent may be a bacterium, for example a probiotic bacterium. The bacterium may be provided as a frozen biomass, freeze-dried, or freeze dried spray dried format (e.g. pellets). Inany embodiment, the probiotic is of the genus Lactobacillus, Bifidobacterium or Saccharomyces. In any embodiment, the composition comprises a prebiotic.

In any embodiment, the ccomposition is free of permeation enhancer, such as chitosan or surfactant-type permeation enhancer. The denatured whey protein acts as a natural permeation enhancer.

In any embodiment, the whey protein is provided by whey protein isolate, whey protein concentrate or milk protein concentrate or isolate.

In any embodiment, at least 50%, 60%, 70%, 80%, 90%, 95%, 99% or all (by weight) of the oral dosage form is constituted by microcapsules.

In any embodiment, the denatured whey protein is gelated.

In any embodiment, the microcapsules are formed by extrusion and gelation in a gelation bath.

In any embodiment, the microcapsules are dried. In any embodiment, the microcapsules are vacuum dried. In any embodiment, the microcapsules are double vacuum dried (e.g. dried by two separate vacuum drying stages).

In any embodiment, the microcapsules are provided as agglomerates of microcapsules. This can be achieved by varying the drying conditions to promote agglomeration during drying. An advantage of agglomerated microcapsules is that they delay the release of active agent contained within the matrix of the individual microcapsules. Once agglomerates reach the ileum, the agglomerate must first break up to release individual microcapsules, before the released microcapsules are broken down to release the encapsulated active agent. This results in a prolonged release profile, which can be tailored by modifying the drying conditions of the microcapsules. Agglomerates of microcapsules are ideal for delivering active agents into the ileum for which a slow prolonged release is desirable.

Drying can be tailored to generated agglomerates using conditions such as vacuum pressure, temperature, agitation, and fill volume. The drying time can be completed within 30 minutes to 12 hours. Drying can occur using vacuum or fluidising conditions.

In any embodiment, the microcapsules comprise as a dry weight %:
about 75 to about 95 % denatured whey protein; and
about 5 to about 25% active agent.

In any embodiment, the microcapsules comprise as a dry weight %:
about 80 to about 90 % denatured whey protein; and
about 10 to about 20 % active agent.

In any embodiment, the microcapsules or agglomerates of microcapsules have an average particle size of 500 to 1000 microns.

In any embodiment, the microcapsules or agglomerates of microcapsules have an average particle size of 200 to 500 or 300 to 500 microns.

In any embodiment, the microcapsules or agglomerates of microcapsules have an average particle size of 500 to 1000 microns.

In any embodiment, the microcapsules or oral dosage form is free of one or all of chitosan, toxin permeation enhancers, surfactant-type permeation enhancer, and cell penetrating peptides.

In any embodiment, the composition is a solid. Examples include tablets, capsules, granules, flakes and powders.

In any embodiment, the composition is a semi-solid, such as a gel

In any embodiment the composition is a liquid, such as a suspension.

In any embodiment, the composition comprises a pharmaceutical excipient.

In any embodiment, the composition comprises a second active agent.

Typically, the whey protein matrix comprises at least 30%, 40%, 50%, 60%, 70%, or 80% β-lactoglobulin (w/w). Suitably, the β-lactoglobulin has a degree of denaturation of at least 50%, 60%, 70%, 80%, 90% or 95%. In a preferred embodiment of the invention, the whey protein comprises at least 70% β-lactoglobulin having a degree of denaturation of at least 90%.

Typically, at least 90% of the microcapsules in the preparation are capable of surviving intact in fresh ex-vivo porcine gastric juice of pH 2.0 for at least three hours at 37° C. under agitation at 150 rpm (employing the methods as described below).

In another aspect, the invention provides an oral dosage form according to the invention, for use in a method of treating a disease or condition in a subject, in which the oral dosage form is administered orally to the subject.

In another aspect, the invention provides a method of making microcapsules comprising the steps of
generating microdroplets comprising denatured protein and active agent (e. probiotic bacteria) by extrusion through a nozzle;
curing the microdroplets by immersion in a curing bath to form microcapsules having a matrix comprising denatured whey protein and active agent contained within the matrix;
removing the microcapsules from the curing bath; and
vacuum drying the microcapsules.

In any embodiment, the drying step comprises double vacuum drying the microcapsules (e.g. vacuum drying the microcapssles in two separate vacuum drying steps).

In any embodiment, the drying conditions are configured to agglomerate the microcapsules during the drying step, and preferably provide agglomerates having an average dimension of 0.5 to 3, more preferably 1 to 2, microns. Drying conditions can be varied to change morphology of particles (i.e. discrete microcapsules or agglomerates of microcapsules). For example, the batch size of micro-capsule size will determine the time of drying and a combination of these factors will generate optimal microcapsules. The creation of agglomerates of microcapsules is in one embodiment defined by the flowrate, speed of agitation and batch size.

In any embodiment, the speed of agitation is the microcapsules during drying is 150 rpm to 800 rpm.

In any embodiment, the temperature during drying is 15° C.- 60° C.

In any embodiment, the flow rate 10 ml/min to 1.5 L/min.

In any embodiment, the microcapsules are vacuum dried for 18-24 hours.

In any embodiment, the microcapsules are vacuum dried to a moisture content of less than 10%, 9%, 8%, 7%, 6% or 5% by weight.

In any embodiment, the microcapsules are vacuum dried to a water activity (Aw) of less than 0.31 (e.g. 0.25 to 0.30), 0.26. 0.21 or 0.16.

In any embodiment, the microcapsules are subjected to a first vacuum drying step and a second vacuum drying step.

In any embodiment, the microcapsules are agitated during the first vacuum drying step.

In any embodiment, the microcapsules are agitated during the second vacuum drying step. In any embodiment, the microcapsules are not agitated during the second vacuum drying step.

In any embodiment, the second vacuum drying step is performed at a pressure lower than the pressure of the first vacuum drying step.

In any embodiment, the first vacuum drying step is performed at a pressure of less than 15 mBar (e.g. 10-15 mBar).

In any embodiment, the second vacuum drying step is performed at a pressure of less than 10 mBar (e.g. 5-10 mBar).

In any embodiment, the microcapsules are dried during the first vacuum drying step to a water activity (Aw) of less that 0.30 (e.g. 0.25 to 0.30) and are dried during the second vacuum drying step to a water activity (Aw) of less that 0.20 (e.g. 0.15 to 0.25).

In any embodiment, the microdroplets are generated using concentric nozzles by extruding a suspension or solution of the active agent (generally including denatured protein) through an inner nozzle and simultaneously extruding a denatured protein suspension through an outer nozzle. This step forms core-shell type microcapsules.

In any embodiment, the microdroplets are generated by extruding a suspension comprising denatured protein and active agent through a single nozzle. This produces microcapsules with a continuous protein matrix and pockets of active agent distributed throughout the matrix.

In any embodiment, the denatured protein comprises or consists of denatured whey protein.

In any embodiment, the active agent is a probiotic bacterium that is frozen, freeze dried, or freeze dried spray dried, in which the probiotic bacterium is hydrated prior to combining with the denatured whey protein. The bacteria may be hydrated in bacterium-specific media or in a liquid comprising milk protein. Hydrating in milk protein has been shown to better supports probiotic stability during cold gelation encapsulation and drying. Milk proteins provide a stabilisation factor for probiotics during the encapsulation process. The milk protein may be milk protein concentrate, skim milk, caseinate, whey protein concentrate or whey protein isolate.

In any embodiment, the whey protein is selected from protein isolate (WPI), whey protein concentrate (WPC), or milk protein isolate (MPI) or concentrate (MPC).

In any embodiment, the suspension comprising denatured whey protein comprises 5 to 20, 10 to 15, 10 to 12 % denatured whey protein by weight.

In any embodiment, the method has an encapsulation efficiency of at least 90%, 95%, 98%, 99% or 99.5% as determined using the method described below.

In any embodiment, the curing bath comprises a citrate buffer. In any embodiment, the curing bath is heated (for example 25-45° C.). In any embodiment, the curing bath has a acidic pH, for example 4.6 to 4.8.

Other aspects and preferred embodiments of the invention are defined and described in the other claims set out below.

### DETAILED DESCRIPTION OF THE INVENTION

All publications, patents, patent applications and other references mentioned herein are hereby incorporated by reference in their entireties for all purposes as if each individual publication, patent or patent application were specifically and individually indicated to be incorporated by reference and the content thereof recited in full.

Definitions and general preferences

Where used herein and unless specifically indicated otherwise, the following terms are intended to have the following meanings in addition to any broader (or narrower) meanings the terms might enjoy in the art:

Unless otherwise required by context, the use herein of the singular is to be read to include the plural and vice versa. The term “a” or “an” used in relation to an entity is to be read to refer to one or more of that entity. As such, the terms “a” (or “an”), “one or more,” and “at least one” are used interchangeably herein.

As used herein, the term “comprise,” or variations thereof such as “comprises” or “comprising,” are to be read to indicate the inclusion of any recited integer (e.g. a feature, element, characteristic, property, method/process step or limitation) or group of integers (e.g. features, element, characteristics, properties, method/process steps or limitations) but not the exclusion of any other integer or group of integers. Thus, as used herein the term “comprising” is inclusive or open-ended and does not exclude additional, unrecited integers or method/process steps.

As used herein, the term “disease” is used to define any abnormal condition that impairs physiological function and is associated with specific symptoms. The term is used broadly to encompass any disorder, illness, abnormality, pathology, sickness, condition or syndrome in which physiological function is impaired irrespective of the nature of the aetiology (or indeed whether the aetiological basis for the disease is established). It therefore encompasses conditions arising from infection, trauma, injury, surgery, radiological ablation, age, poisoning or nutritional deficiencies.

As used herein, the term “treatment” or “treating” refers to an intervention (e.g. the administration of an agent to a subject) which cures, ameliorates or lessens the symptoms of a disease or removes (or lessens the impact of) its cause(s) (for example, the reduction in accumulation of pathological levels of lysosomal enzymes). In this case, the term is used synonymously with the term “therapy”.

Additionally, the terms “treatment” or “treating” refers to an intervention (e.g. the administration of an agent to a subject) which prevents or delays the onset or progression of a disease or reduces (or eradicates) its incidence within a treated population. In this case, the term treatment is used synonymously with the term “prophylaxis”.

As used herein, an effective amount or a therapeutically effective amount of an agent defines an amount that can be administered to a subject without excessive toxicity, irritation, allergic response, or other problem or complication, commensurate with a reasonable benefit/risk ratio, but one that is sufficient to provide the desired effect, e.g. the treatment or prophylaxis manifested by a permanent or temporary improvement in the subject’s condition. The amount will vary from subject to subject, depending on the age and general condition of the individual, mode of administration and other factors. Thus, while it is not possible to specify an exact effective amount, those skilled in the art will be able to determine an appropriate “effective” amount in any individual case using routine experimentation and background general knowledge. A therapeutic result in this context includes eradication or lessening of symptoms, reduced pain or discomfort, prolonged survival, improved mobility and other markers of clinical improvement. A therapeutic result need not be a complete cure. Improvement may be observed in biological / molecular markers, clinical or observational improvements. In a preferred embodiment, the methods of the invention are applicable to humans, large racing animals (horses, camels, dogs), and domestic companion animals (cats and dogs).

In the context of treatment and effective amounts as defined above, the term subject (which is to be read to include “individual”, “animal”, “patient” or “mammal” where context permits) defines any subject, particularly a mammalian subject, for whom treatment is indicated. Mammalian subjects include, but are not limited to, humans, domestic animals, farm animals, zoo animals, sport animals, pet animals such as dogs, cats, guinea pigs, rabbits, rats, mice, horses, camels, bison, cattle, cows; primates such as apes, monkeys, orangutans, and chimpanzees; canids such as dogs and wolves; felids such as cats, lions, and tigers; equids such as horses, donkeys, and zebras; food animals such as cows, pigs, and sheep; ungulates such as deer and giraffes; and rodents such as mice, rats, hamsters and guinea pigs. In preferred embodiments, the subject is a human. As used herein, the term “equine” refers to mammals of the family Equidae, which includes horses, donkeys, asses, kiang and zebra.

As used herein, the term “oral dose form” refers to a composition formulated for oral administration. Examples include tablets, pills, capsules, thin films, pastes, gels, powders, granules, liquid solutions or suspension. Tablets may be formed by direct compression. The oral dosage form generally includes an active agent and a pharmaceutical excipient. In one aspect of the present invention, the active agent is provided in the form of a microcapsule, in which the active agent is contained within a matrix configured to protect the active agent during gastric transit and release the active agent in the ileum. To this end, the matrix may comprise gelated denatured protein such as whey protein. In one embodiment the oral dosage form is provided as a unit dose, containing a single dose of active agent. In the case of diabetes for example, this may be 10-100 IU of insulin.

The term “microcapsule” as used herein should be understood to mean a particle comprising an active component encapsulated within a matrix comprising denatured whey protein. Preferably, the microcapsule has an average diameter (average particle size) of 200 to 1000 microns. Size is determined using a method of laser diffractometery (Mastersizer 2000, Stable Micro Systems, Surrey, UK). This method is determines the diameter, mean size distribution and D (v, 0.9) (size at which the cumulative volume reaches 90% of the total volume), of micro-encapsulates with diameters in the range of 0.2-2000 µm. For insulin microcapsule size analysis, micro-encapsulate batches were resuspended in Milli-Q water and size distribution is calculated based on the light intensity distribution data of scattered light. Measurement of microencapsulate size is performed at 25° C. and six runs are performed for each replicate batch (Doherty et al., 2011) (Development and characterisation of whey protein micro-beads as potential matrices for probiotic protection,S.B. Doherty, V.L. Gee, R.P. Ross, C. Stanton, G.F. Fitzgerald, A. Brodkorb, Food Hydrocolloids Volume 25, Issue 6, August 2011, Pages 1604-1617). A preferred method of producing the microcapsules in by extrusion through a nozzle, typically a vibrating nozzle, and curing (gelation) in a gelation bath. In one embodiment, the suspension is sprayed through a nozzle and laminar break-up of the sprayed jet is induced by applying a sinusoidal frequency with defined amplitude to the spray nozzle. Examples of vibrating nozzle machines are and EnCapsulator (Inotech, Switzerland), or equivalent scale-up version such as those produced by Brace GmbH or Capsulae and the like. Typically, the spray nozzle has an aperture of between 100 and 600 microns, preferably between 150 and 400 microns, suitably about 300 microns. Suitably, the frequency of operation of the vibrating nozzle is from 900 to 4000 Hz. Generally, the electrostatic potential between nozzle and acidification bath is 0.85 to 1.8 V. Suitably, the amplitude is from 4.7 kV to 7 kV. Typically, the falling distance (from the nozzle to the curing bath) is less than 100 cm, preferably less than 80 cm, suitably between 50 and 70 cm, preferably between 45 and 65 cm, and ideally about 55 cm. The flow rate of suspension (passing through the nozzle) is typically from 3.0 to 120 ml/min; an ideal flow rate is dependent upon the nozzle size utilized within the process.

“Cold-gelated” as applied to microcapsules refers to microcapsules having a gelated protein matrix formed by extrusion through a nozzle and curing (gelation) is a gelation bath. Cold gelated microcapsules may be extruded through a single nozzle (in which the matrix contains pockets of active agent) or through a double concentric nozzle which form core-shell type microcapsules. Method of producing microcapsules by cold-gelation is described in WO2010/119041, WO2014/198787 and WO2016/096929.

“Gastro-resistant”: means that the microencapsulates can survive intact for at least 60 minutes in the simulated stomach digestion model described in Minekus et al., 1999 and 2014 (A computer-controlled system to simulate conditions of the large intestine with peristaltic mixing, water absorption and absorption of fermentation product, Minekus, M., Smeets-Peeters M, Bernalier A, Marol-Bonnin S, Havenaar R, Marteau P, Alric M, Fonty G, Huis in’t Veld JH, Applied Microbiology Biotechnology. 1999 Dec;53 (1):108-14) and (Minekus et al., 2014, A standardised static in vitro digestion method suitable for food - an international consensus, Minekus, A. et al., Food Function, 2014, 5, 1113).

“Ileal-sensitive”: means that the microencapsulates are capable of releasing their contents in vivo in the ileum of a mammal.

“Encapsulation efficiency” means the amount of active agent loaded into the microcapsule carrier. The Encapsulation efficiency is calculated as follows by determining the free insulin concentration, and the total amount of insulin (Initial insulin concentration).

$$\text{EE \%} \text{=} \text{100} \text{-} \left[\left(\text{Free insulin conc} \text{.} / \text{Initial insuling conc} \text{.}\right) \times 100\right]$$

“Vacuum drying” is the mass transfer operation in which the moisture present in a substance, usually a wet solid, is removed by means of creating a vacuum. In chemical processing industries like food processing, pharmacology, agriculture, and textiles, drying is an essential unit operation to remove moisture. Vacuum drying is generally used for the drying of substances which are hygroscopic and heat sensitive, and is based on theprinciple of creating a vacuum to decrease the chamber pressure below the vapor pressure of the water, causing it to boil. With the help of vacuum pumps, the pressure is reduced around the substance to be dried. This decreases the boiling point of water inside that product and thereby increases the rate of evaporation significantly. The result is a significantly increased drying rate of the product. The pressure maintained in vacuum drying is generally 0.03-0.06 atm and the boiling point of water is 25-30° C. The vacuum drying process is a batch operation performed at reduced pressures and lower relative humidity compared to ambient pressure, enabling faster drying. “Water activity” (aw) is the partial vapor pressure of water in a substance divided by the standard state partial vapor pressure of water. It is measured by the method described in Carter, B. P., Galloway, M. T., Campbell, G. S., & Carter, A. H. (2015). The critical water activity from dynamic dewpoint isotherms as an indicator of pre-mix powder stability. Journal of Food Measurement and Characterization, 9(4), 479-486. The operator’s manual of the equipment used is provided at http://manuals.decagon.com/Manuals/13893_AquaLab%20Pre_Web.pdf Values for water activity (Aw) provided herein are obtained at 25° C. unless stated otherwise.

The present invention also provides pharmaceutical compositions. Such compositions comprise an effective amount of microcapsules or agglomerates according to the invention, and a pharmaceutically acceptable excipient or carrier. In a specific embodiment, the term “pharmaceutically acceptable” means approved by a regulatory agency of the Federal or a state government or listed in the U.S. Pharmacopeia or other generally recognized pharmacopeia for use in animals and more particularly in humans. The term “excipient” refers to a diluent, adjuvant, excipient, or vehicle with which the microcapsules or agglomerates of the invention are administered. Such pharmaceutical excipients can be sterile liquids, such as water and oils, including those of petroleum, animal, vegetable or synthetic origin, such as peanut oil, soybean oil, mineral oil, sesame oil and the like. Water is a preferred carrier when the pharmaceutical composition is administered intravenously. Saline solutions and aqueous dextrose and glycerol solutions can also be employed as liquid carriers, particularly for injectable solutions. Suitable pharmaceutical excipients include starch, glucose, lactose, sucrose, gelatin, malt, rice, flour, chalk, silica gel, sodium stearate, glycerol monostearate, talc, sodium chloride, dried skim milk, glycerol, propylene glycol and water.

### EXEMPLIFICATION

The invention will now be described with reference to specific Examples. These are merely exemplary and for illustrative purposes only: they are not intended to be limiting in any way to the scope of the monopoly claimed or to the invention described. These examples constitute the best mode currently contemplated for practicing the invention.

### Probioitic Microcpaules - Single Stage Drying

Hydrate Whey Protein powder (9.0% - 11.0% protein content) in water Hydrate and measure pH
Denature protein at 95.5 DegC for 80-85 seconds Cool to room temperature to 22 DegC for 16 - 20 hours Measure level of protein agglomeration by HPLC
Hydrate probiotics in milk powder or designated media for specified strain powder or designated media for specified strain
Add probiotic to denatured protein (1:5 or 1:9 or 1:20)
Extrude solution through a single or double nozzle If double nozzle is used, outer nozzle is pure denature protein (9% solids)
Polymerise microcapsules in sodium citrate buffer Ph 4.6 - 4.8 at 20 - 30 DegC
Allow solution to polymerise for max 4 hours at RT in citrate buffer
Wash micro-capsules in water and in skim milk powder solution (5-20% solids content
Dry the material under vacuum at room temperature for 18 20 hours Measure moisture (<10% and Aw content (<0.3) Quantify probiotic content as per usual method
Store at room temperature or refrigerated temperature

### Probiotic Micropcaules - Double Stage Drying

Hydrate Whey Protein powder (10.0% - 11.0% protein content) in water Hydrate and measure pH
Denature protein at 95.5 DegC for 80-85 seconds Cool to room temperature to 22 DegC for 16 -20 hours Measure level of protein agglomeration by HPLC
Hydrate probiotics in milk powder or designated media for specified strain
Add probiotic to denatured protein (1:5 or 1:9 or 1:20)
Extrude solution through a single or double nozzle If double nozzle is used, outer nozzle is pure denature protein (9% solids)
Polymerise microcapsules in todium citrate buffer Ph 4.5 - 4.8 at 20 - 30 DegC
Allow solution to polymerise for max 4 hours at RT in citrate buffer
Wash micro-capsules in water and in skim milk powder solution (5-20% solids content
Dry the material under vacuum at room temperature for 18 20 hours
Transfer material to secondary vacuum chamber for secondary drying
Dry for further 12-38 hour drying at <10 mBar (2nd stage drying) with / without agitation Measure moisture (<5% and Aw content (<0.2) Quantify probiotic content as per usual method
Store at room temperature or refrigerated temperature

### EQUIVALENTS

The foregoing description details presently preferred embodiments of the present invention. Numerous modifications and variations in practice thereof are expected to occur to those skilled in the art upon consideration of these descriptions. Those modifications and variations are intended to be encompassed within the claims appended hereto.

1. A composition comprising gastric-resistant ileal-sensitive microcapsules comprising a matrix and active agent contained within the matrix, in which the matrix comprises denatured whey protein, in which the microcapsules are cold-gelated and vacuum dried microcapsules.

2. A composition according to claim 1 having a water activity (Aw) of less than 0.30.

3. A composition according to claim 1 having a water activity (Aw) of less than 0.25.

4. A composition according to any preceding Claim, in which the active agent comprises probiotic bacteria.

5. A composition claims 4, in which the microcapsules comprise as a % dry weight:
80 to 90% denatured whey protein; and
10 to 20% probiotic bacteria.

6. A composition of any of claims 1 to 5, in which the microcapsules have an average particle size of 200 to 500 microns.

7. A method of making microcapsules comprising the steps of
providing microdroplets comprising denatured whey protein and active agent by extrusion;
curing the microdroplets by immersion in a curing bath to form microcapsules having a matrix comprising denatured whey protein and active agent contained within the matrix;
removing the microcapsules from the curing bath; and
vacuum drying the microcapsules.

8. A method of claim 7, in which the active agent comprises probiotic bacteria.

9. A method of claim 7 or 8, in which the microcapsules are vacuum dried to a water activity of 0.20 or less.

10. A method of claim 7 or 8, in which the microcapsules are vacuum dried to a water activity of 0.15 or less.

11. A method of any of claims 7 to 10, in which the microcapsules are subjected to a first vacuum drying step and a second vacuum drying step.

12. A method according to claim 11, in which the microcapsules are agitated during the first vacuum drying step and optionally not agitated during the second drying step.

13. A method according to claim 11 or 12, in which the first vacuum drying step is performed at a pressure of 10-15 mBar.

14. A method according to claim 13, in which the second vacuum drying step is performed at a pressure of 5-10 mBar.
